(19) United States
(12) Patent Application Publication
(10) Pub. No.: US 2010/0130858 A1
(43) Pub. Date: May 27, 2010

(54) Puncture Treatment Supporting Apparatus

(76) Inventors: Osamu Arai, Tokyo (JP); Takao Iwasaki, Miyagi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/089,231

(22) PCT Filed: Oct. 6, 2006
(86) PCT No.: PCT/JP2006/320055

§ 371 Date: Apr. 4, 2008

(30) Foreign Application Priority Data
Oct. 6, 2005 (JP) 2005-293103

(51) Int. Cl. A61B 8/14

(52) U.S. Cl. 600/443

(57) ABSTRACT

[Problems] To provide a puncture treatment supporting apparatus enabling puncture treatment by previously performing puncture simulation and enabling its treatment evaluation.

[Means for Solving Problems] A puncture treatment supporting apparatus comprises an ultrasonic probe, ultrasonic image construction means for constructing an ultrasonic image, volume data storing means for storing volume data on a medical image diagnostic apparatus, probe position/direction detecting means for detecting the position and direction of the ultrasonic probe, tomographic image construction means for constructing tomographic image having the same cross section as the ultrasonic image by using the information on the position and direction of the ultrasonic probe, display means for displaying these images, and puncture means for inserting a puncture needle.

The apparatus, further comprises simulation image constructing means for constructing a simulation image by adding a puncture guideline indicating the puncture position and direction of the puncture needle to the tomographic image, and the display means displays the simulation image along with the ultrasonic image.

### TECHNICAL FIELD

The present invention relates to a puncture treatment supporting apparatus for drawing up a puncture operation plan by simulation and displaying the puncture operation proceedings.

### BACKGROUND ART

Ultrasonic diagnostic apparatuses obtain a tomographic image of soft tissues in a living body using ultrasonic pulse reflection method. Ultrasonic diagnostic apparatuses are compact in size and inexpensive compared to other medial image diagnostic apparatuses, highly safe since there is no exposure to radiation such as X-rays, and has features such as being capable of imaging blood flow, thus are widely used, for example, in digestive organs departments, urology departments, and obstetrics and gynecology departments.

The ultrasonic diagnostic apparatus is used for inserting a puncture needle into an object while observing an ultrasonic image, obtaining a part of tumor cells for a sample using the puncture needle or cauterizing the tumor using an RF coil provided at the end point of the puncture needle. The probes to be used at this time are a biopsy type provided with a groove for inserting a puncture needle to a part of an array-type probe, or an adapter type that is an array-type probe in which an adapter for puncture is mounted.

In Patent Document 1, a technique is disclosed for constructing and displaying a tomographic image corresponding to an ultrasonic image in accordance with the position and the angle of a probe arbitrarily specified by an operator, from volume data regarding an object collected by a medical image diagnostic apparatus other than an ultrasonic diagnostic apparatus (such as an X-ray CT apparatus or an MRI apparatus).

While the operator develops a puncture plan in advance by imaging the position of a tumor in the body of an object, it is difficult to do so and to perform a puncture operation when the target region for the treatment is in a complicated region in the body of the object.

The objective of the present invention is to provide a puncture treatment supporting apparatus capable of performing a puncture simulation in advance, performing a puncture treatment in accordance with the simulation, and evaluating the treatment.

Patent Document 1: JP-A-2002-112998

### DISCLOSURE OF THE INVENTION

### Problems to be Solved

In order to achieve the above-described objective, the puncture treatment supporting apparatus of the present invention comprising:

an ultrasonic probe for transmitting/receiving ultrasonic waves to/from an object;

ultrasonic image construction means for constructing an ultrasonic image on the basis of the ultrasonic signals obtained by the ultrasonic probe;

volume data storing means for storing volume data of the object being imaged by a medical image diagnostic apparatus;

probe position/direction detecting means for detecting the position and direction of the ultrasonic probe;

tomographic image construction means for constructing from the volume data a tomographic image having the same cross-section as the ultrasonic image, using information on the position and direction of the ultrasonic probe;

display means for displaying the ultrasonic image and the tomographic image; and

puncture means for inserting a puncture needle into the object via the ultrasonic probe,

is characterized in comprising:

simulation image construction means for constructing a simulation image provided with a puncture guideline indicating the position and direction for inserting the puncture needle on the tomographic image,

wherein the display means displays the simulation image along with the ultrasonic image.

Also, the simulation image construction means synthesizes the puncture guidelines and the volume data, stores the synthesized data in the volume data storing means, and constructs the simulation image from the synthesized volume data using information on the position and direction of the ultrasonic probe.

Also, the simulation image construction means has:

puncture guideline creating means for creating the puncture guideline using the ultrasonic image or the tomographic image; and

volume data synthesizing means for synthesizing and storing the volume data and the puncture guideline,

and constructs a simulation image provided with the puncture guideline, from the synthesized volume data.

### BRIEF DESCRIPTION OF THE DIAGRAMS

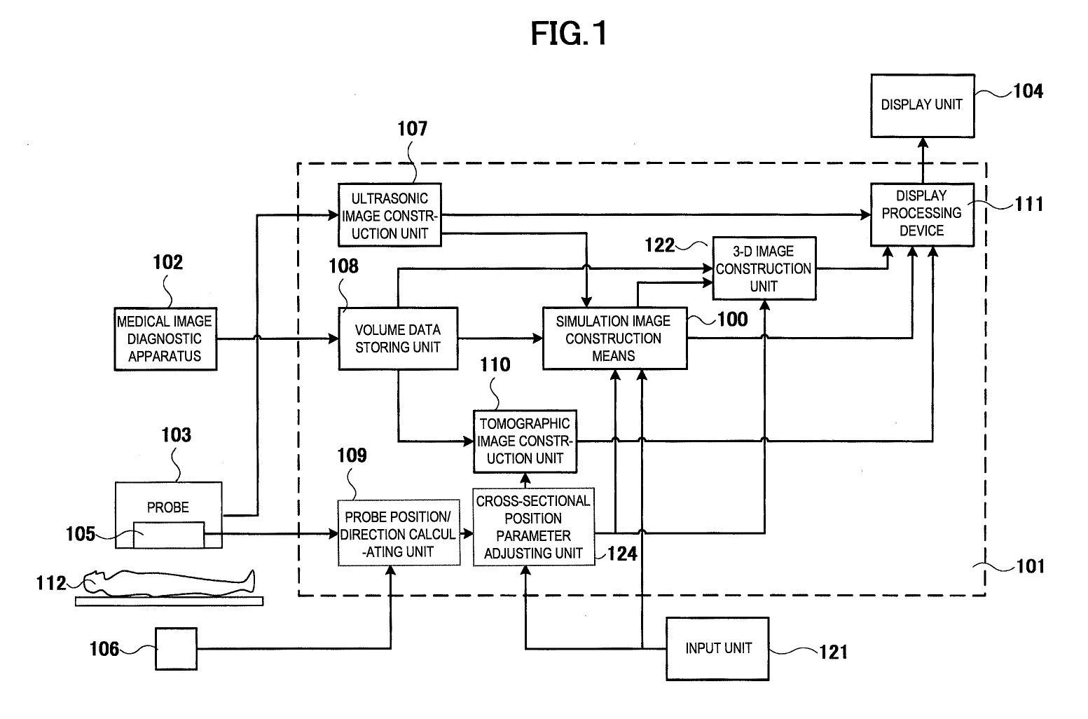

FIG. 1 shows a system configuration of the puncture treatment supporting apparatus related to the present invention.

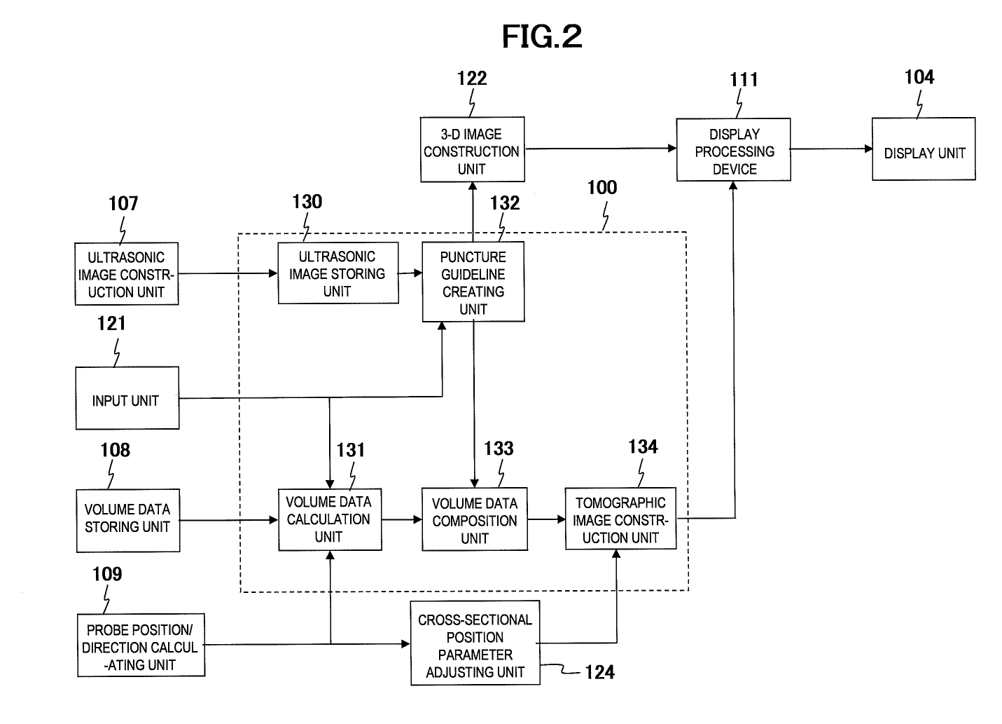

FIG. 2 shows the detail of the puncture treatment supporting apparatus related to the present invention.

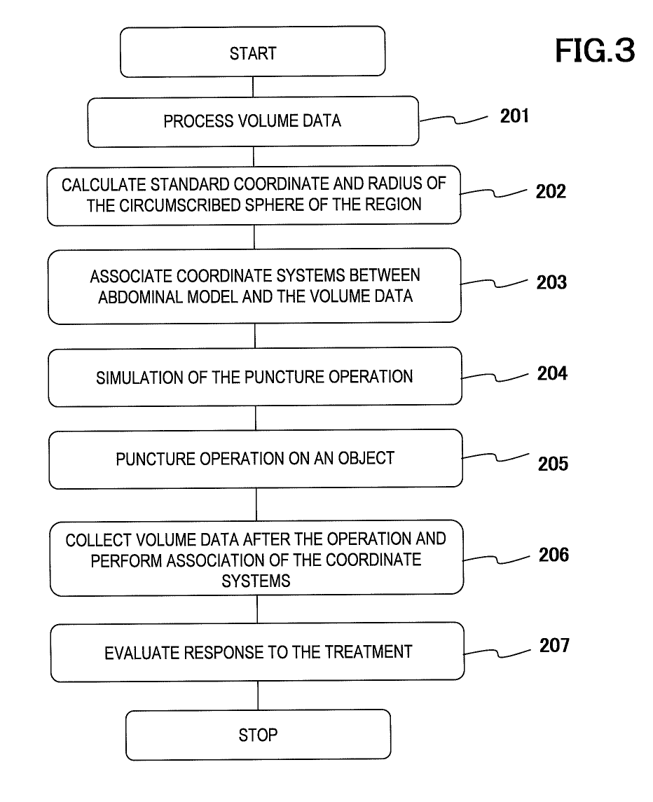

FIG. 3 shows the operation procedure of the present invention.

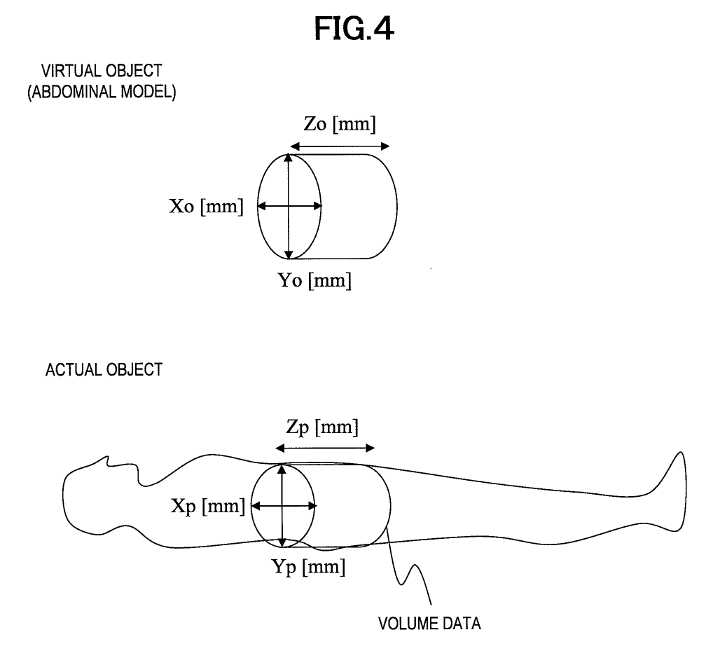

FIG. 4 shows the concept of a scale conversion related to the present invention.

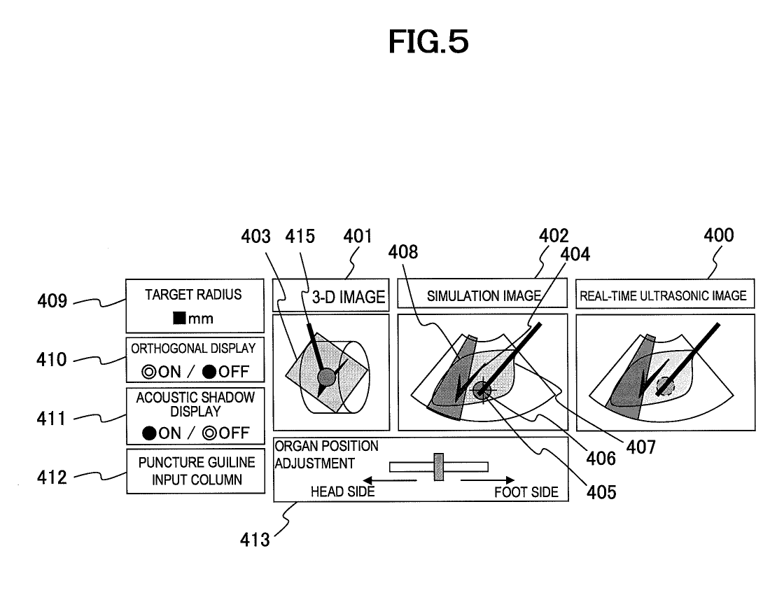

FIG. 5 shows a display example related to the present invention.

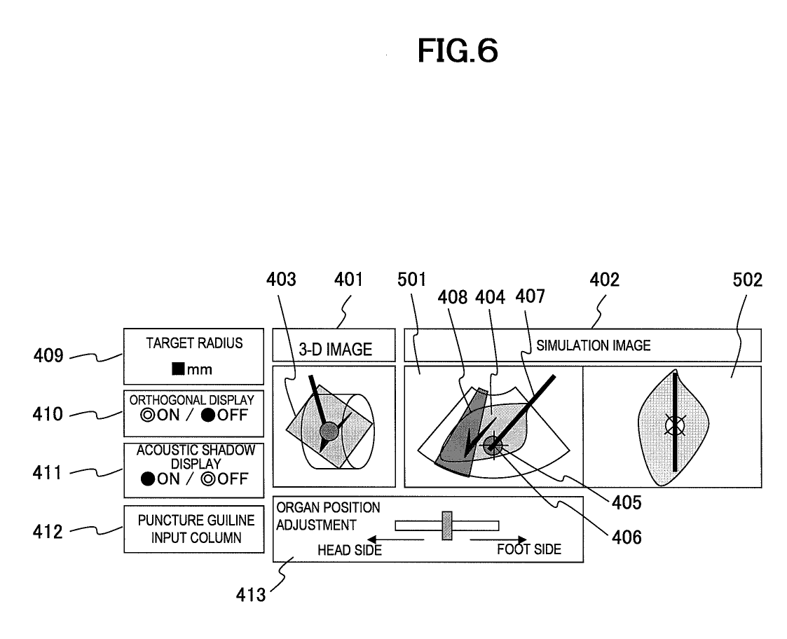

FIG. 6 shows a display example related to the present invention.

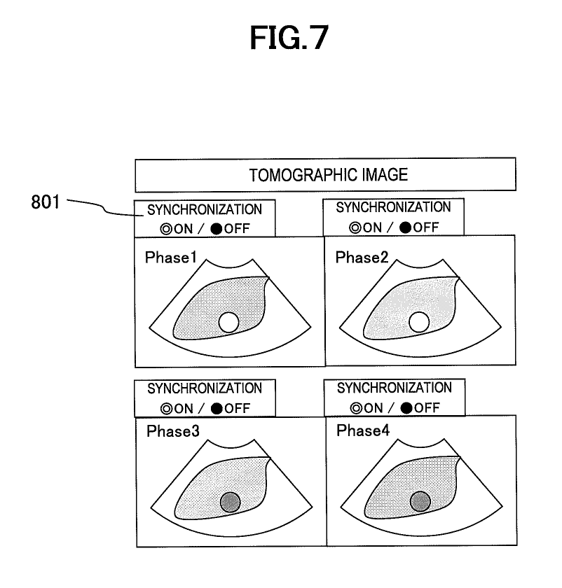

FIG. 7 shows a display example related to the present invention.

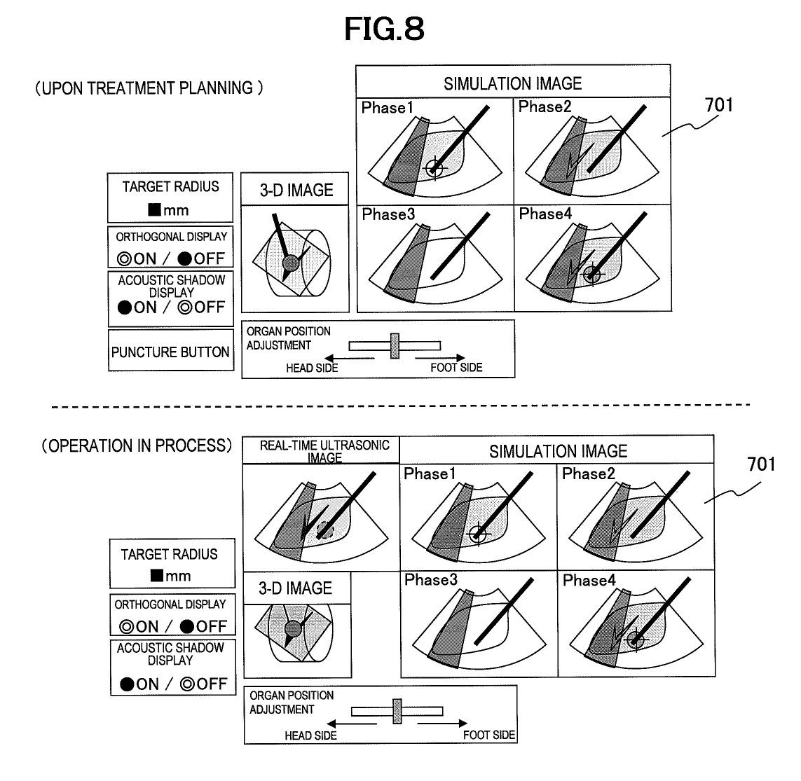

FIG. 8 shows a display example related to the present invention.

### BEST MODE FOR CARRYING OUT THE INVENTION

Hereinafter, the system configuration of the puncture treatment supporting apparatus related to the present invention will be described using FIG. 1.

The puncture treatment supporting apparatus comprises:

a medical image diagnostic apparatus 102 such as an X-ray CT apparatus or an MRI apparatus;

a probe 103 for transmitting/receiving ultrasonic waves to/from an object 112;

a probe position sensor 105 formed together with the probe 103;

a source 106 placed in the vicinity of the object 112, and

for detecting the movement of the probe position sensor 105 by a magnetic field, etc.;

an image processing device 101 for imaging the image data obtained from the medical image diagnostic apparatus 102 or the probe 103; and

a display unit 104 for displaying the image processed in the image processing device 101.

Further, a central control device (not shown in the diagram) is provided in the image processing device 101, and controls the respective components in the image processing device 101.

Next, inner structure of the image processing device 101 will be described. The image processing device 101 is mainly formed by:

a first route for constructing an ultrasonic image on the basis of the echo signals outputted from the probe 103;

a second route for constructing a 3-dimensional image using the volume data outputted from the medical image diagnostic apparatus 102;

a third route for constructing a tomographic image having the same cross-section as the above-mentioned ultrasonic image using the volume data outputted from the medical image diagnostic apparatus 102; and

a fourth route for constructing a puncture simulation image using the volume data outputted from the medical image diagnostic apparatus 102. A display processing device 111 performs processing of juxtaposing and displaying, or superimposing and displaying the images that are constructed in the respective routes, and displays the processed images.

Also, a process for detecting the position and direction of the probe 103 using the probe position sensor 105 and the source 106 will be described. The probe position sensor 105 detects magnetic signals generated from the source 106 to a 3-dimensional space. Then the position and direction of the probe position sensor 105 in a reference coordinate system formed by the source 106 are transmitted to the probe position/direction calculating unit 109. The probe position/direction calculating unit 109 calculates a scan plane coordinate of the ultrasonic image from the transmitted position and direction.

Here, the first route for constructing an ultrasonic image will be described. The probe 103 is for transmitting/receiving ultrasonic waves to/from the object 112, and has a plurality of transducers for generating ultrasonic waves and receiving echo signals. An ultrasonic image construction unit 107 converts the echo signals transmitted from the probe 103 into digital signals, and creates ultrasonic image data such as a black and white tomographic image (B-mode image) or a color flow mapping image (CFM image). The ultrasonic image data created in the ultrasonic image construction unit 107 are outputted to a display processing device 111. The ultrasonic image constructed in the ultrasonic image construction unit 107 and displayed is a tomographic image.

Next, the second route for constructing a 3-dimensional image using the volume data of the medical image diagnostic apparatus will be described. The second route has a volume data storing unit 108 being connected to the medical image diagnostic apparatus 102 using a network, etc. and is for storing the volume data, and a 3-dimensional image construction unit 122 for creating 3-dimensional image data from the stored volume data using a method such as the volume rendering method. The 3-dimensional image data created in the 3-dimensional image construction unit 122 are outputted to the display processing device 111.

Also, the 3-dimensional image construction unit 122 corresponds the coordinate of the volume data to the scan plane coordinate of the probe 103 detected in the probe position/direction calculating unit 109, synthesizes a scan plane guide of the same cross-sectional position of the ultrasonic image to the 3-dimensional image data, and outputs the synthesized data. A scan plane guide is displayed on the 3-dimensional image displayed on the display unit 104. Accordingly, an operator can recognize the cross-sectional position of the ultrasonic image 3-dimensionally.

Next, the third route for constructing a tomographic image having the same cross-section as the ultrasonic image using the volume data outputted from the medical image diagnostic apparatus 102 will be described. The third route has a volume data storing unit 108 for storing volume data, and a tomographic image construction unit 110 arranged by being connected to a cross-sectional position parameter adjusting unit 124 which is connected to the probe position/direction calculating unit 109, and is for creating tomographic image data having the same cross-section position as the ultrasonic image imaged at the position of the probe 103 by an X-ray CT apparatus or an MRI apparatus. In concrete terms, the tomographic image construction unit 110 calculates the scan plane coordinate in the coordinate system of the tomographic image based on the scan plane coordinate calculated in the probe position/direction calculating unit 109. Then the tomographic image construction unit 110 creates tomographic image data with respect to the part that the volume data and the scan plane are superimposed, from the coordinate data of the scan plane in the coordinate system of the volume data and the rotation angle around the XYZ-axis of the scan plane. The created tomographic image data are outputted to the display processing device 111.

Next, the fourth route for creating the puncture simulation image using the volume data outputted from the medical image diagnostic apparatus 102 will be described.

The fourth route has a simulation image construction unit 100 for creating the puncture simulation image data using the volume data stored in the volume data storing unit 108. The simulation image construction unit 100 will be described using FIG. 2.

The simulation image construction unit 100 has:

a volume data calculating unit 131 for performing the scaling or position adjustment of the volume data outputted from the volume data storing unit 108;

an ultrasonic image storing unit 130 for storing the ultrasonic image obtained in the ultrasonic image construction unit 107;

a puncture guideline creating unit 132 for creating the puncture guideline using the positional information obtained from an input device 121 or the ultrasonic image storing unit 130;

a volume data synthesizing unit 133 for synthesizing the processed volume data and the puncture guideline; and

a tomographic image construction unit 134 for creating the simulation image data having the same cross-sectional position as the ultrasonic image being imaged in the position and direction of the probe 103.

The tomographic image construction unit 134 calculates the scan plane coordinate in the coordinate system of the tomographic image based on the scan plane coordinate calculated by the probe position/direction calculating unit 109. Then the tomographic image construction unit 134 creates the simulation image data with regard to the part that the volume data and the scan plane are synthesized, from the coordinate data of the scan plane in the coordinate system of the synthesized volume data and the rotation angle around the XYZ-axis of the scan plane.

A plurality of memories are mounted in the volume data storing unit 108, and are capable of storing a plurality of volume data. Therefore, the plurality of volume data obtained in different times can be stored.

Next, operation procedure of the present invention will be described using the flow chart in FIG. 3.

### (Step 201)

First, the volume data calculating unit 131 converts the volume data into the data of a blood vessel, tumor, bone or air that is being enhanced. Generally, the volume data of an X-ray CT apparatus or an MRI apparatus being collected after injecting contrast medium are collected in a plurality of phases after injecting the contrast medium, and the enhanced region is different in each phase. For example, in the case of a liver, while a tumor is enhanced and is visualized in clarity in an arterial phase wherein the most contrast medium flows in an artery, the blood vessel becomes difficult to see. On the other hand, while the tumor is hard to see in a portal wherein the most contrast medium flows in the portal vein, the blood vessel can be clearly identified.

The volume data calculating unit 131 processes the volume data stored in the volume data storing unit 108 using a method such as a threshold value method or region growing method, with respect to each phase so that more range of the blood vessel, tumor, bone or air can be identified. Any method can accurately extract the region in the case that the difference in luminance is clear between the respective tissues and the surrounding tissues. In the case that the region cannot be extracted, the operator may extract it using the input device 121. Then the volume data calculating unit 131 processes the volume data by coloring the region of the extracted blood vessel or tumor, etc.

### (Step 202)

Next, the volume data calculating unit 131 calculates the standard coordinate and the radius of a circumscribed sphere in the region extracted by the volume data. The method for calculating the standard coordinate and the radius of the circumscribed sphere will be omitted since it is a commonly known technique. The central position of the circumscribed sphere is the position to be a target for the puncture operation. Also, the size of the radius is to be an index for determining the cauterization time upon performing the radiofrequency ablation.

### (Step 203)

Next, the association of the coordinate systems is performed between an abdominal model of a human body to be used in place of an object upon simulation and the volume data created by the volume-data calculating unit 131.

First, calibration is performed to make the standard coordinate (or the original point of the coordinate) of the abdominal model coincides with the standard coordinate (or the original point of the coordinate) of the volume data, using the abdominal model of the human body to be used in place of the object. Then the direction for setting the abdominal model is adjusted so that the coordinate system of the abdominal model is coincided with the direction of a unit vector of the coordinate system of the volume data.

In the case that there is a difference of body size between the abdominal model and the object to be the target for the puncture operation stored in the volume data, the volume data calculating unit 131 performs scale conversion between the coordinate system of the abdominal model and the coordinate system of the volume image data. A scale conversion matrix is used to perform the scale conversion.

Here, a conceptual diagram of the scale conversion is shown in FIG. 4. In FIG. 4, the diagram shown in the upper side illustrates an abdominal model of a human body formed approximately in circular cylinder, and the diagram shown in the lower side illustrates the volume data of the abdomen in the object obtained by an X-ray CT apparatus or an MRI apparatus.

First, a horizontal width Xo, a vertical width Yo and a length Zo in the body axial direction of a waste line of the abdominal model are inputted to the volume data calculating unit 131 using the input device 121. Next, the volume data calculating unit 131 extracts the body surface of the volume data using a method such as the threshold method, and calculates a horizontal width Xp, a vertical width Yp and a length Zp in the body axial direction. Then using the above-calculated data, the following matrix is created and set as a scale conversion matrix S.

$$S = [ \begin{matrix}Xp / Xo & 0 & 0 & 0 \\ 0 & Yp / Yo & 0 & 0 \\ 0 & 0 & Zp / Zo & 0 \\ 0 & 0 & 0 & 1\end{matrix} ] \qquad [ Formula 1 ]$$

The calibration correction data and the scale conversion matrix S obtained by the calibration above are to be stored in the volume data calculating unit 131.

In this way, the volume data calculating unit 131 is capable of coinciding the standard coordinates of the abdominal model of the human body and the volume data. Also, adjustment can be made even when there is a difference in body size between the abdominal model and the object.

### (Step 204)

Next, the simulation of the puncture operation is performed applying the probe 103 onto the abdominal model. The operator sets a cross mark on the central position of the tumor using the input device 121, while observing whether the central position of the tumor is included in the cross-section of the simulation image being calculated by the tomographic image construction unit 134. Then the volume data calculating unit 131 adds the cross mark data at the position of the volume data corresponding to the cross mark set in the simulation image.

Next, the operator determines the position and direction for inserting the puncture needle while observing the simulation image, and creates the puncture guidelines by the puncture guideline creating unit 132. In concrete terms, the operator specifies two points on a 3-dimensional image 401 or a simulation image 402 displayed on the display unit 104 using the input device 121, and the positional information having the two specified points as the end is inputted to the puncture guideline creating unit 132. Then the puncture guideline creating unit 132 creates the puncture guideline (position, length and direction) from the specified two points of the positional information. The puncture guideline created in the puncture guideline creating unit 132 is converted into a 3-dimensional coordinate, and stored in the volume data synthesizing unit 133 in a form corresponded to the volume data.

Also, as another method, the puncture guideline creating unit 132 creates a puncture guideline using luminance information of the ultrasonic image. It is created by inserting the puncture needle into the abdominal model, imaging an ultrasonic image which includes the puncture needle using the probe 103, and storing the imaged ultrasonic image in the ultrasonic image storing unit 130. The puncture guideline creating unit 132 creates the puncture guideline using the luminance information of the stored ultrasonic image.

Here, a method for creating the puncture guideline using luminance information of the ultrasonic image will be described in detail. In the abdominal model, the outward form is a simulated abdomen of a human body, and the inside thereof has a homogeneous material and tenderness being similar to the abdomen of a human body. The ultrasonic image imaged while the probe 103 is applied onto the abdomen model is homogeneous and low in luminance. In the case of inserting the puncture needle into the abdomen model, the puncture needle in the ultrasonic image has high luminance. Given this factor, the puncture guideline creating unit 132 creates a binary image by binarizing the ultrasonic image by high luminance and low luminance. Then the puncture guideline creating unit 132 creates a puncture guideline (position, length and direction) extracted as a high luminance part of the binary image as, for example, a green colored image data. The detected puncture guideline is converted into a 3-dimensional coordinate, and stored in the volume data synthesizing unit 133 in a form being corresponded to the volume data. By using the volume data synthesized with the guideline by the volume data synthesizing unit 133, a simulation data having the same cross-sectional position as the ultrasonic image imaged in the same position and direction as the probe 103 is created. Accordingly, the state of the puncture needle being inserted is displayed on the simulation image as a green colored image.

FIG. 5 is a display example of the display unit 104. The display unit 104 displays an ultrasonic image 400 created in the above-described first route, a 3-dimensional image 401 created in the above-described second route, and a simulation image 402 created in the above-described fourth route. A scan guide 403 indicates the cross-sectional position of the simulation image 402 being calculated by the tomographic image construction unit 134. The display processing device 111 can select and display these images. On the simulation image 402, a blood vessel 404, a tumor 405, a central position of the tumor 406 and a puncture guideline 407 for determining the position and direction to insert the puncture needle, and an acoustic shadow 408 indicating a part wherein the ultrasonic image can not be constructed very well due to the bone or air in the body of the object, are displayed. Also, the display unit 104 displays a column 409 for displaying the radius of a tumor which is a target, a button 410 for selecting whether to display the cross-section orthogonal to the simulation image or not, a button 411 for selecting whether to display an acoustic shadow 408 or not, a puncture guideline inputting column 412 for inputting the puncture guideline to display the place for performing the puncture by the angle with respect to the probe 103, etc., and a scroll bar 413 for inputting how to move the position of an organ in accordance with breathing of the object. Input information of the above-mentioned buttons and columns are given by the input device 121. The conventional technique in regard to the acoustic shadow is disclosed in WO2004/0984141A1.

At this time, as shown in FIG. 5, the tumor 405, the blood vessel 404 or the acoustic shadow 408 are displayed on the simulation image 402. In the case of creating the guideline 407 using the input device 121, the operator needs to pay attention to, for example:
(a) set the tumor 405 or the blood vessel 404 etc. not to be hidden by the acoustic shadow 408,
(b) set the puncture guideline 407 to pass through the central point of the tumor 405, and
(c) set the puncture guideline 407 not to pass through the blood vessel 404.

As mentioned above, the puncture needle can be inserted into the abdomen model by which the object is simulated, having the simulation image 402 as a guide. The operator can perform puncture on the model as if performing it on the actual object. This is also useful for the puncture training for an inexperienced operator.

Also, the puncture guideline data detected in a puncture guideline creating unit 132 is converted into a 3-dimensional coordinate, and the 3-dimensional image construction unit 122 constructs a 3-dimensional image data provided with a puncture guideline 415. In the 3-dimensional image 401 displayed on the display unit 104, the body surface is displayed translucently, and the blood vessel 404, the tumor 405, the scan plane guide and the puncture guideline 415 are 3-dimensionally displayed on the inside of the body.

In puncture treatment, while the position of the bone does not move but organs move by breathing of the object, and the position of the acoustic shadow varies accordingly. Given this factor, in the present step, the cross-sectional position parameter adjusting unit 124 changes the cross-sectional position of the simulation image 402 created by the tomographic image construction unit 134, by sliding the scroll bar 513 in FIG. 5 using the input device 121. For example, by sliding a sliding bar 413 coordinating with the breathing, the tomographic image construction unit 134 can change the cross-sectional position of the simulation image 402. Also, by setting the sliding bar 413 using the input device 121 so as to periodically repeat parallel translation, for example, once in every 10 seconds, the tomographic image construction unit 134 can periodically change the cross-sectional position of the simulation image 402.

Also, in the present step, as shown in FIG. 6, the puncture guideline cross-sectional image 502 including the puncture guideline 407 can be displayed which is the orthogonal cross-section to the simulation image 501 on the left side. In concrete terms, the cross-sectional position parameter adjusting unit 124 adjusts the cross-sectional position parameter so as to rotate the volume data having the puncture guideline 407 as the central axis, based on the position and direction of the puncture guideline 407. The tomographic image construction unit 134 constructs a puncture guideline cross-sectional image 502 using the adjusted cross-sectional position parameter and the volume data. In addition, while the volume data is rotated having the puncture guideline 407 as the central axis, the central axis of the probe 103 or the blood vessel 404 may also be used.

### (Step 205)

Next, in step 204, the operator performs the puncture operation while observing the set puncture guideline 407.

The ultrasonic image constructed in the first route is a real-time ultrasonic image 400 obtained in real time. The simulation image 402 constructed in the fourth route is the same cross-section as the real-time ultrasonic image 400 and includes the puncture guideline 407. The operator secures the probe 103, confirming that the puncture guideline 407 is displayed on the simulation image 402 toward the direction of the central position of the tumor, or that the puncture guideline 407 is displayed from a start-point to an end-point. Then the operator inserts the puncture needle into the object while observing the ultrasonic image 400, and secures the puncture needle upon reaching the central position of the tumor 405. The operator then performs the operation such as collecting a part of the tumor cells or cauterizing the tumor using an RF coil provided at the end of the puncture needle.

### (Step 206)

After the puncture operation, volume image data after the treatment are obtained. The volume data before the treatment and the volume data after the treatment are stored in the volume data storing unit 108. Then the tomographic image construction unit 110 creates the tomographic data using each volume data, and displays them on the display unit 104.

Here, the operator searches for, for example, a bifurcation of a blood vessel in the vicinity of the tumor, and specifies the reference point using the input device 121. The volume data storing unit 108 compares the tomographic images created by the volume data before and after the treatment, and the respective coordinates are made to correspond to each other on the basis of the reference point. Conversion matrix M expressing the relative positional relationship between the volume data before the treatment and after the treatment is calculated according to the following formula.

$$M = [ \begin{matrix}1 & 0 & 0 & dX \\ 0 & 1 & 0 & dY \\ 0 & 0 & 1 & dZ \\ 0 & 0 & 0 & 1\end{matrix} ] \qquad [ Formula 2 ]$$

Here, both of the volume data are assumed to be imaged from the direction of the object. The present formula uses the parallel translation model, and dX, dY and dZ are the values to be calculated based on the reference-point coordinate of the volume image data before the treatment and the volume image data after the treatment. In this way, the coordinate system between the volume data are corresponded to each other in the volume data storing unit 108 using the conversion matrix M. Then the tomographic image construction unit 110 constructs two tomographic images from the two volume data so as to synchronize with the position of the probe 103, and displays them on the display unit 104.

Here, a method for constructing two tomographic images using two volume data obtained at the different times will be concretely described. The operator moves the probe 103 so that a specified cross-sectional image of the object is displayed on the first tomographic image being constructed using the first volume data. Then the operator turns off a synchronization button using the input device 121 at a point that the specified cross-sectional image is displayed on the first tomographic image on the display unit 104. When the synchronization button is turned off, the cross-sectional position parameter adjusting unit 124 stops to transmit the information on the position and direction of the probe 103 calculated by the probe position/direction calculating unit 109 to the tomographic image construction unit 110. Consequently, the first tomographic image constructed in the tomographic image construction unit 110 stops operating in compliance with the movement of the probe 103, and the first tomographic image comes to rest at the state on which the specified cross-sectional image is being displayed.

Also, the operator moves the probe 103 so that the specified cross-sectional image of the object is displayed on the second tomographic image being constructed using the second volume data. This specified cross-section is the same as the specified cross-section being displayed on the first tomographic image. Then the operator turns off the synchronization button using the input device 121 when the specified cross-sectional image is displayed on the second tomographic image on the display unit 104. When the synchronization button is turned off, the cross-sectional position parameter adjusting unit 124 stops to transmit the information on the position and direction of the probe 103 calculated by the probe position/direction calculating unit 109 to the tomographic image construction unit 110. Consequently, the second tomographic image constructed by the tomographic image construction unit 110 stops operating in compliance with movement of the probe 103, and the second tomographic image comes to rest at the state on which the specified cross-sectional image is being displayed.

Then the operator moves the probe 103 so that the specified cross-section displayed on the first cross-sectional image and the second cross-sectional image are to be displayed on the ultrasonic image constructed in the first route. When the specified cross-section is displayed on the ultrasonic image, the probe 103 is fixed, and the respective synchronization buttons are turned on using the input device 121.

The cross-sectional position parameter adjusting unit 124 transmits the information on the position and direction of the probe 103 calculated by the probe position/direction calculating unit 109 to the tomographic image construction unit 110. Then the tomographic image construction unit 110 constructs the first tomographic image and the second tomographic image having the same cross-sectional position as the ultrasonic image being imaged in the position and direction of the probe 103, using the first volume data and the second volume data. Accordingly, the first tomographic image and the second tomographic image can be juxtaposed and displayed on the display unit 104. It can be displayed in the same manner in the case of having three or more volume image data.

FIG. 7 shows a display example of the display unit 104 in the present step. An synchronization button 801 is a button for the operator to set the synchronized condition or non-synchronized condition. Four tomographic images are to be created here using four volume data acquired at different times. The operator makes only one tomographic image being deviated from the specified image including the bifurcation of the blood vessel, etc. out of the four displayed tomographic images to be synchronized with the probe 103, and makes the other three tomographic images to remain in non-synchronized condition. Then the probe 103 is moved to the position at which the same cross section as the specified image of the tomographic image synchronizing with the probe 103 is displayed. The probe 103 is then secured, the synchronization button is turned on using the input device 121, and the cross-sectional position parameter adjusting unit 124 sets the four tomographic images in the synchronized condition. Accordingly, the cross-sectional position parameter adjusting unit 124 is capable of adjusting the cross-sectional position by selecting one tomographic image out of four tomographic images.

As mentioned above, the present invention is capable of performing the coordination of a plurality of tomographic images. The coordination of the coordinate system can be independently performed with respect to each volume data, thus the present invention can be applied to the case that the number of volume data is increased. In clinical practice, a plurality of tomographic images are often juxtaposed and displayed using the volume data obtained in arterial phase before the treatment, portal phase before the treatment, arterial phase after the treatment and portal phase after the treatment.

FIG. 8 shows another display example of the display unit 104 in the present step. While the plurality of tomographic images are displayed in the display pattern of FIG. 7, a plurality of simulation images may also be displayed in the same manner. In concrete terms, the operator moves the probe 103 so that the puncture guideline 407 is displayed on the simulation image constructed using each of the volume data. Then the operator turns off the synchronization button using the input device 121 when the puncture guideline 407 is displayed on the display unit 104. When the synchronization button is turned off, the cross-sectional position parameter adjusting unit 124 stops to transmit the information on the position and direction of the probe 103 calculated by the probe position/direction calculating unit 109 to the tomographic image construction unit 134. Consequently, the first tomographic image constructed by the tomographic image construction unit 134 stops operating in compliance with the movement of the probe 103, and the first tomographic image comes to rest at the state on which the specified cross-sectional image is being displayed. Accordingly, as shown in FIG. 8, the display unit 104 is capable of displaying the simulation images 701 having the different time phases, upon drawing out the treatment plan or during the operation.

### (Step 207)

The operator can compare the tomographic image before the treatment and the tomographic image after the treatment displayed on the same cross-section, and evaluate the treatment effect on the puncture operation cross-section. If the tomographic images before and after the treatment are superimposed and displayed, correspondent relationship between the treated area and non-treated area can be easily recognized. As for the superimposing method, variety of methods such as translucent synthesis by the alpha blending method and a method for extracting contours and synthesizing can be used. If it is judged that the treatment is insufficient in the treatment judgment process, it will be useful to extract the region to be retreated on the CT volume image data and stored the data, for performing retreatment by going back to step 201.

The present information is not intended to be limited in the above embodiments, and various changes may be made without departing from the scope of the invention. For example, in step 204, imaging and displaying the ultrasonic image of the abdominal model is not fundamental. In the case of not imaging the ultrasonic image of the abdominal model, the position or direction for inserting the puncture guideline may be inputted on the display screen using the input device 121.

1. A puncture treatment supporting apparatus comprising:
an ultrasonic probe for transmitting/receiving ultrasonic waves to/from an object;
ultrasonic image construction means for constructing an ultrasonic image based on an ultrasonic signal obtained by the ultrasonic probe;
volume data storing means for storing the volume data of the object being imaged by a medical image diagnostic apparatus;
probe position/direction detecting means for detecting the position and direction of the ultrasonic probe;
tomographic image construction means for constructing, from the volume data, a tomographic image having the same cross-section as the ultrasonic image, using information on the position and direction of the ultrasonic probe;
display means for displaying the ultrasonic image and the tomographic image; and
puncture means for inserting a puncture needle into the object through the ultrasonic probe,
characterized in comprising simulation image construction means for constructing a simulation image wherein the puncture guideline for indicating the position and direction for inserting the puncture needle is provided on the tomographic image,
wherein the display means displays the simulation image along with the ultrasonic image.

2. The puncture treatment supporting apparatus according to claim 1, wherein the simulation image construction means synthesizes the puncture guideline with the volume data, makes the volume data storing means to store the synthesized data, and constructs the simulation image from the synthesized volume data using information on the position and direction of the ultrasonic probe.

3. The puncture treatment supporting apparatus according to claim 1, wherein the simulation image construction means has:
puncture guideline creating means for creating the puncture guideline using the ultrasonic image or the tomographic image; and
volume data synthesizing means for synthesizing the puncture guideline with the volume data, and storing the synthesized data,
characterized in constructing a simulation image provided with the puncture guideline, from the synthesized volume data.

4. The puncture treatment supporting apparatus according to claim 1, wherein the display means displays the simulation image and the ultrasonic image, juxtaposing them on the same screen.

5. The puncture treatment supporting apparatus according to claim 1, characterized in comprising 3-dimensional image construction means for constructing a 3-dimensional image using the volume data, wherein the display means displays the simulation image and the 3-dimensional image, juxtaposing them on the same screen.

6. The puncture treatment supporting apparatus according to claim 1, wherein:
the volume data storing means stores a plurality of volume data acquired at each different times; and
the tomographic image construction means constructs a plurality of tomographic images corresponding to the plurality of volume data.

7. The puncture treatment supporting apparatus according to claim 1, wherein:
the volume data storing means stores the plurality of volume data respectively acquired in different times; and
the simulation image construction means constructs the simulation image corresponding to the plurality of volume data.

8. The puncture treatment supporting apparatus according to claim 6 or 7, wherein the display means displays the tomographic images or the plurality of simulation images, juxtaposing them on the same screen.

9. The puncture treatment supporting apparatus according to claim 6 or 7, wherein the plurality of volume data are the volume data obtained before the treatment and the volume data obtained after the treatment.

10. The puncture treatment supporting apparatus according to claim 1, characterized in comprising cross-sectional position parameter adjusting means for changing the cross-sectional position of the simulation image constructed using the volume data.

11. The puncture treatment supporting apparatus according to claim 1, wherein the simulation image construction means causes the display means to display the puncture guideline cross-sectional image that is an orthogonal cross-section to the simulation image.

12. The puncture treatment supporting apparatus according to claim 3, characterized in comprising a model of a predetermined region of the object, wherein the puncture guideline creating means creates the puncture guideline using the ultrasonic image or the tomographic image obtained in the condition that the ultrasonic probe is applied on the model.

13. The puncture treatment supporting apparatus according to claim 3, wherein the puncture guideline creating means specifies the position and direction of the puncture guideline by specifying a straight line on the ultrasonic image or the tomographic image displayed on the display means.

14. The puncture treatment supporting apparatus according to claim 3, wherein the puncture guideline creating means specifies the position and direction of the puncture needle using luminance information of the ultrasonic image including the puncture needle.

15. The puncture treatment supporting apparatus according to claim 3, wherein the puncture guideline creating means binarizes the high luminance region and the low luminance region of the ultrasonic image respectively, and causes the binarized images to be displayed on the simulation image.

16. The puncture treatment supporting apparatus according to claim 1, characterized in comprising volume data calculating means for adjusting the displacement of the size or position of the volume data, wherein the simulation image construction means constructs the simulation image on the basis of the adjusted volume data.
